(19) United States
(12) Patent Application Publication
(10) Pub. No.: US 2015/0044313 A1
(43) Pub. Date: Feb. 12, 2015

(54) LACTIC ACID BACTERIA CULTURE OF MUNG BEAN AND THE PREPARATION METHOD OF THE SAME, AND THE COSMETIC COMPOSITION COMPRISING THE SAME

(71) Applicants: Doosan Corporation, Seoul (KR); Biovan Co., Ltd., Seoul (KR)

(72) Inventors: Chang-Seo PARK, Gwacheon-city (KR); Sang-June NAM, Seongnam-city (KR); Wang-Keun CHOI, Hwasung-city (KR); Yu-Ryang PYUN, Seoul (KR); Hyung-Yong CHO, Jeonjoo-si (KR); Seok-Cheol CHO, Seoul (KR); Moo-Chang KOOK, Seoul (KR); Chang-Woo LEE, Seoul (KR); So-Young CHUNG, Yongin-city (KR)

(21) Appl. No.: 14/525,661

(22) Filed: Oct. 28, 2014

Related U.S. Application Data
(63) Continuation of application No. 11/994,866, filed on Sep. 18, 2008.

(30) Foreign Application Priority Data
Jul. 7, 2005 (KR) 10-2005-0061136

(51) Int. Cl. A61K 36/48; A61K 31/197; A61K 8/97; A61K 8/44; A61Q 19/00; A61Q 19/08

(52) U.S. Cl. CPC A61K 36/48; A61Q 19/00; A61Q 19/08; A61K 8/97; A61K 8/44; A61K 31/197; A61K 2800/805
USPC 424/757

(57) ABSTRACT

Disclosed is a lactic acid bacteria culture of mung bean obtained by culturing lactic acid bacteria in a culture medium containing mung bean extract. The culture contains the mung bean extract and GABA (&gammad; Aminobutyric acid), so that it exhibits effects of promoting collagen synthesis and alleviating inflammation. Accordingly, the cosmetic composition containing the culture can be usefully used as a cosmetic composition for promoting collagen biosynthesis, preventing or improving skin senescence, ant i-inflammatory and preventing or improving skin injury.

### TECHNICAL FIELD

The invention relates to a lactic acid bacteria culture obtained by culturing lactic acid bacteria in a culture medium comprising the mung bean extract and GABA, a method of preparing the culture, a cosmetic composition containing the same and a method of preparing the cosmetic composition.

### BACKGROUND ART

GABA (γ-Aminobutyric acid) is non-protein amino acid and is well known as a main inhibitory neurotransmitter of a central nervous system of an animal. In the animals, the GABA is known to participate in many physiological mechanisms to activate the blood stream of a brain and to increase the air supply, thereby promoting a metabolic function of brain cells. In addition, it is also known that the GABA participates in regulations of secretion of prolactin and growth hormone and has effect of decreasing the blood pressure and alleviating pains. Accordingly, the GABA arouses pharmacological interests.

Owing to the functions, the GABA can be used as functional food material and raw material of cosmetics.

In recent years, it is also known peroxisome proliferators activated receptors (PPAR) existing in skin constituting cells, which PPAR plays a very important role in expressions of injury and inflammation curing processes of skin.

Specifically, the PPAR is a factor of regulating an energy homeostasis, and in particular, participates in regulations of permeability of skin barrier and regulations of skin state such as suppression of multiplication of epidermal layer and induction of differentiation of epidermal layer, through various mechanisms. Owing to the features, the PPAR serves as a core regulator of various skin diseases such as psoriasis due to overgrowth of the epidermal layer, injury cure and acne as well as skin diseases related to inflammation.

Accordingly, although it is not much known a specific signal transfer of a material contributing to the homeostasis of the skin, it is attempted a research on functions of phospholipid relating to the PPAR which is recently known. The PPAR is known to have three sub-types and it is found that PPAR a has a possibility of a receptor of phosphatidylserine [Michalik et al., The Journal of Cell Biology, 154, pp 799-814, 2001].

It is actually acknowledged that when clofibrate and WY14643 which are agonists of PPAR α are applied to the skin, the inflammation due to stimulus materials is decreased [Sheu et al., The Journal of investigative Dermatology, 118, pp 94-101, 2002].

Up to date, although materials such as glucocorticoid have been used as anti-inflammatory agent, they exhibit chronic side effects when they are continuously administrated or treated, so that an immunological reaction is decreased and thus there occurs limitations in the treatment. Thereby, it has been considered that the agonist of PPAR α is a local and effective treatment method, as compared to the glucocorticoid.

In the mean time, secretion of interleukin-6 (IL-6) which is cytokine is increased due to TNF-α which is peculiarly reacted with external antigens and the increased interleukin-6 causes inflammatory reactions. Thus, it can be considered that the decrease of expressions of the interleukin-6 is an index of suppressing the anti-inflammatory and hyperimmune reactions.

According to prescriptions related to skin cosmetic, which is disclosed in documents such as Donguibogam (Exemplar of Korean Medicine), the mung bean (Phaseolus aureus, Phaseolus radiatus) exhibits skin cosmetic effects. In particular, it is also known that mung bean protein and mung bean flavonoid are effective in the cleansing and vitexin and isovitexin, which are physiological activating ingredients, have an effect of preventing skin senescence due to its excellent antioxidant effect.

### DISCLOSURE

### Technical Problem

Accordingly, the inventors conducted various tests for constituting a culture medium so as to develop a method capable of producing the GABA in a high concentration. As a result, the inventors confirmed that when lactic acid bacteria are cultured using mung bean as a main raw material of the culture medium, it can be obtained a culture containing the GABA as well as excellent ingredients originated from the mung bean, in high amounts. In addition, during investigation of various efficacies of the culture containing the mung bean extract and GABA in a high amount, using the mung bean, it was confirmed that the culture promotes collagen synthesis of the skin, comprises a material acting as a ligand of PPAR α of a tissue cell in the skin, thereby alleviating an inflammatory reaction of the skin due to external stimulus and decreasing expression of interleukin-6 relating to anti-inflammatory and hyperimmune reactions.

An object of the invention is to provide a lactic acid bacteria culture of mung bean containing mung bean extract and GABA and a method of preparing the same.

Another object of the invention is to provide a cosmetic composition containing the lactic acid bacteria culture of mung bean containing the mung bean extract and GABA, as an effective ingredient, and a method of preparing the cosmetic composition.

Still another object of the invention is to provide a cosmetic composition for promoting collagen biosynthesis, preventing or improving skin senescence, anti-inflammatory and preventing or improving skin injury.

### Technical Solution

In order to achieve the above objects, there is provided a lactic acid bacteria culture of mung bean obtained by culturing lactic acid bacteria in a culture medium containing the mung bean, the composition containing mung bean extract and GABA (γ-Aminobutyric acid).

According to an embodiment of the invention, the lactic acid bacteria culture of mung bean is obtained by seeding and culturing the lactic acid bacteria in a culture medium including the mung bean extract.

According to an embodiment of the invention, the lactic acid bacteria culture of mung bean is obtained by seeding and culturing the lactic acid bacteria in a culture medium including the mung bean extract and monosodium glutamate (MSG).

According to an embodiment of the invention, the mung bean extract is extracted with water.

According to an embodiment of the invention, the lactic acid bacteria are lactic acid bacteria capable of expressing glutamate dicarboxylase to convert the MSG into the GABA, preferably Lactobacillus sakei or Lactobacillus brevis.

According to another aspect of the invention, there is provided a method of preparing a lactic acid bacteria culture of mung bean obtained by culturing lactic acid bacteria in a culture medium containing the mung bean, the method comprising steps of: (a) preparing mung bean extract; (b) preparing a culture medium including the mung bean extract obtained in the step (a) and monosodium glutamate (MSG); (c) seeding lactic acid bacteria in the culture medium prepared in the step (b), the bacteria being capable of converting the MSG into GABA (γ-Aminobutyric acid) by glutamate dicarboxylase; and (d) culturing the lactic acid bacteria seeded in the step (c) to prepare a lactic acid culture of mung bean containing the mung bean extract and the GABA.

According to an embodiment of the invention, an addition amount of the MSG in the step (b) is 1 to 30 wt % for a total weight of the mung bean extract.

According to another aspect of the invention, there is provided a method of preparing a cosmetic composition containing a lactic acid bacteria culture of mung bean obtained by culturing lactic acid bacteria in a culture medium containing the mung bean, the method comprising steps of: removing a bacterial cell and an insoluble ingredient from the lactic acid bacteria culture of mung bean containing the mung bean extract and GABA, the culture being prepared according to the method of preparing the lactic acid bacteria culture of mung bean; and mixing a sub-material with the culture from which the bacterial cell and the insoluble ingredient are removed in the preceding step.

In addition, the invention provides a cosmetic composition containing a lactic acid bacteria culture of mung bean containing mung bean extract and GABA, as an effective ingredient.

According to an embodiment of the invention, the lactic acid bacteria culture of mung bean is prepared by the method of preparing the lactic acid bacteria culture of mung bean.

According to an embodiment of the invention, the lactic acid bacteria are lactic acid expressing glutamate dicarboxylase to convert MSG into GABA.

According to an embodiment of the invention, the cosmetic composition is prepared by the method of preparing the cosmetic composition containing the lactic acid bacteria culture of mung bean.

According to an embodiment of the invention, the cosmetic composition is a composition for promoting collagen biosynthesis.

According to an embodiment of the invention, the cosmetic composition is a composition for preventing or improving skin senescence.

According to an embodiment of the invention, the cosmetic composition is an anti-inflammatory composition.

According to an embodiment of the invention, the cosmetic composition is a composition for preventing or improving skin injury.

### Advantageous Effects

The lactic acid bacteria culture of mung bean containing the mung bean extract and GABA of the invention is able to exhibit the effect of preventing or improving the skin senescence through the collagen synthesis promotion. In addition, it can exhibit an anti-inflammatory effect and an effect of alleviating or improving the skin stimulus. Accordingly, the lactic acid bacteria culture of mung bean containing the mung bean extract and GABA can be usefully used as the cosmetic composition for promoting collagen biosynthesis, preventing or improving skin senescence, anti-inflammatory and preventing or improving skin injury.

### DESCRIPTION OF DRAWINGS

FIG. 1 is a schematic view showing a method of preparing a lactic acid bacteria culture of mung bean using the mung bean, according to an embodiment of the invention;

FIG. 2 shows a measurement result of a GABA content in a lactic acid bacteria culture of mung bean prepared in an embodiment 2, using a HPLC;

FIG. 3 shows a measurement result of a collagen synthesis promoting efficacy of a lactic acid bacteria culture of mung bean of the invention;

FIG. 4 shows a measurement result of an inflammation alleviating efficacy by TPA (12-0-tetradecanonylphorbol-13-acetate) of a lactic acid bacteria culture of mung bean of the invention;

FIG. 5 is a photograph showing an inflammation alleviating efficacy of a lactic acid bacteria culture of mung bean of the invention, and

FIG. 6 shows a measurement result of an effect of a lactic acid bacteria culture of mung bean of the invention on secretion of interleukin-6 (IL-6) related to anti-inflammatory and hyperimmune reactions.

### BEST MODE

Hereinafter, the invention will be specifically described.

During research on a method capable of producing GABA in a high concentration using lactic acid bacteria, the inventors found out that when the lactic acid bacteria producing the GABA are seeded and cultured using mung bean extract as a raw material of a culture medium, it can be obtained a culture including effective ingredients of the mung bean, the GABA and various peptide ingredients produced by the lactic acid bacteria. In addition, it was confirmed that if solid and pigment ingredients are removed from the produced culture and sub-materials are added so as to use the culture as a raw material of cosmetics, since it is exhibited effects of promoting collagen synthesis and alleviating inflammation, the culture can be used as the raw material of cosmetics for the senescence prevention and anti-inflammatory.

In addition, the inventors could obtain a natural material having merits of existing mung bean extract and lactic acid bacteria culture by successfully culturing Lactobacillus Sakei B2-16 (Deposit No. KFCC-11321) as the lactic acid bacteria, which is a lactic acid bacteria strain capable of producing GABA screened from Kimchi, (refer to Korean Patent Application No. 2003-5828), using culture medium ingredients of a raw material of mung bean.

A method of preparing a lactic acid bacteria culture of mung bean containing mung bean extract and GABA of the invention is as follows.

The lactic acid bacteria culture of mung bean obtained by culturing the lactic acid bacteria in a culture medium containing the mung bean can be obtained by a method comprising steps of (a) preparing mung bean extract; (b) preparing a culture medium including the mung bean extract obtained in the step (a) and monosodium glutamate (MSG); (c) seeding lactic acid bacteria in the culture medium prepared in the step (b), the bacteria being capable of converting the MSG into GABA (γ-Aminobutyric acid) by glutamate dicarboxylase; and (d) culturing the lactic acid bacteria seeded in the step (c) to prepare a lactic acid culture of mung bean containing the mung bean extract and the GABA.

In the step (a), it is advantageous to continuously supply the mung bean for obtaining the mung bean extract from a same area. Although there is a small difference between production yields depending on production areas, the production areas little affect final qualities of fermented products.

In addition, the mung bean is preferably washed before it is used as an ingredient of the culture medium, so as to remove agricultural chemicals remaining on a surface thereof. In the washing process, cold water below 30° C. is preferably used and it is advantageous to carry out the process in a short time such as 30 minutes or less, so as to prevent water soluble nutrients from being lost. When the temperature of washing water is above 30° C. and the washing time exceeds 30 minutes, the nutrients of the mung bean can be lost.

Furthermore, peeled or non-peeled mung bean can be used as the mung bean used as the culture medium, which little affects the final product.

The washed mung bean is advantageously pulverized for an extracting process. However, non-pulverized mung bean can be used for the extracting process of the invention.

The mung bean extract included in the culture medium can be obtained by using a variety of extraction solvents known in the art. At this time, the usable extraction solvent can be at least one selected from a group consisting of water, absolute or water-containing low alcohol having a carbon number of 1 to 4 such as methanol, ethanol, butanol and propanol, mixing solvent of water and the low alcohol, acetone, ethyl acetate, chloroform, butyl acetate and 1,3-butylene glycol. In addition, water is preferably used. That is, water is the best solvent, in consideration of stability of final product and culture of lactic acid bacteria. Further, it is possible to obtain the extract suitable for the invention using water.

An amount of the extract solvent used for obtaining the mung bean extract is 2 to 20 times as much as the mung bean, preferably 5 to 15 times. When the amount of the extraction solvent is too little, an extract efficiency is lowered. To the contrary, when the amount of the extraction solvent is too much, a concentration of effective ingredient after the extraction is low. Accordingly, when the effective ingredient is used as a fermentation source, a conversion rate of GABA is lowered.

In addition, an extraction temperature of the mung bean extract is preferably 40˜70° C. When the extraction temperature is low, it takes much time to extract water soluble nutrients, and when the temperature is above 70° C., starch of the mung bean is gelatinized, so that it is difficult to separate the usable ingredients.

Additionally, extraction time of the mung bean extract is preferably 3 to 24 hours, more preferably 6 to 18 hours. Even more preferably, the mung bean extract is extracted for 6 to 18 hours while being stirred at 30 to 100 rpm. When the extraction time is too short, a concentration of the nutrient is low and when the extraction time is too long, it can be a burden on the process.

The obtained mung bean extract is subject to a centrifugal separation to remove solid ingredients thereof, so that a supernatant solution is obtained, which is then added as nutrients of a culture medium for producing the GABA. At this time, the solid ingredients may be removed after the culturing.

In the step (b), the culture medium includes the mung bean extract and MSG.

In addition, according to a preferred embodiment of the invention, the culture medium of the invention includes carbon source, nitrogen source, micro elements, surfactant or a mixture thereof, as well as the mung bean extract and MSG. More preferably, the culture medium includes the carbon and nitrogen sources. Even more preferably, the culture medium includes the carbon and nitrogen sources, and micro elements or surfactant.

In the culture medium of the invention, the carbon source is at least one selected from a group consisting of glucose, sucrose, maltose, fructose, lactose, xylose, galactose, arabinose and a combination thereof, preferably a group consisting of sucrose, fructose, glucose, galactose, arabinose and lactose, more preferably a group consisting of sucrose, fructose and glucose.

An amount of the carbon source used is preferably 1 to 20 wt % for a total weight of the mung bean extract, more preferably 2 to 10 wt %, and even more preferably 3 to 6 wt %.

In the culture medium of the invention, the nitrogen source is at least one selected from a group consisting of yeast extract, soytone, peptone, beef extract, trypton, casitone and a combination thereof, preferably a group consisting of yeast extract, peptone, trypton and soytone, more preferably yeast extract or soytone.

An amount of the nitrogen source used is preferably 1 to 20 wt % for a total weight of the mung bean extract, more preferably 1 to 10 wt % and even more preferably 2 to 4 wt %.

In the culture medium of the invention, the micro element is at least one selected from a group consisting of magnesium sulfate, sodium sulfate, manganic sulfate, ferric sulfate, calcium chloride and a combination thereof.

An amount of the micro element used is preferably 0.001 to 1 wt % for a total weight of the mung bean extract, more preferably 0.01 to 1 wt % and even more preferably 0.03 to 0.3 wt %.

One feature of the invention is that the surfactant can be used as the micro element. In case that the surfactant is used, it is possible to eliminate the necessity of the micro element in the culture medium. The surfactant used for the invention is at least one non-ionic surfactant selected from a group consisting of poly alcohol fatty acid such as monostealysineglycerine, polyethyleneglycol fatty acid such as stearic acid polyoxyl, polyoxyethylene alcohol such as raulmacrogol, sorbitan fatty acid such as oleic acid sorbitan, polyoxyethylene sorbitan fatty acid such as polysorbate and a combination thereof, more preferably polyoxyethylene sorbitan fatty acid and most preferably polysorbate.

In case that the culture medium is made by adding the non-ionic surfactant, the lactic acid bacteria can be normally cultured without the micro element. Accordingly, it is possible to eliminate the necessity of the micro element in the culture medium.

An amount of the surfactant used as the micro element is preferably 0.01 to 1 wt % for the total weight of the mung bean extract, more preferably 0.05 to 0.3 wt %.

In addition, in the step (b), an addition amount of the MSG is 1 to 30 wt % for the total weight of the mung bean extract, more preferably 1 to 20 wt %, and even more preferably 1 to 15 wt %. When the addition amount of the MSG is too much, the MSG which is not converted into the GABA remains on a final product. When the addition amount of the MSG is too little, the content of the GABA is too little in the final product.

The culture medium having all the ingredients (mung bean extract, MSG and other ingredients) mixed therein is preferably subject to a sterilizing treatment. At this time, the sterilizing treatment is preferably carried out at 60 to 121° C. for 15 to 30 minutes, so as to minimize destruction of the nutrients. When the sterilizing treatment is carried out at a lower temperature or for a shorter time, the sterilization effect is reduced. When the sterilizing treatment is carried out at a higher temperature or for a longer time, the nutrients are destructed too much.

In the step (c), the lactic acid bacteria are seeded in the culture medium sterilized in the step (b).

In the method of the invention, glutamate decarboxylase which is expressed in the lactic acid bacteria participates in the conversion of the MSG into GABA.

Although the lactic acid bacteria used for the invention are not particularly limited as long as the bacteria express the glutamate decarboxylase, the bacteria are preferably Lactobacillus genus strain, most preferably Lactobacillus sakei or Lactobacillus brevis, more preferably at least one selected from a group consisting of Lactobacillus sakei B2-16 (Deposit No. KFCC-11321), Lactobacillus brevis B1-14, Lactobacillus brevis B1-31, Lactobacillus brevis B2-22, Lactobacillus brevis B2-27, Lactobacillus brevis B2-29, Lactobacillus brevis B3-18, Lactobacillus brevis B3-25, Lactobacillus brevis B3-30 and Lactobacillus brevis A128, which are recorded as GABA producing strains in a Korean Patent Application No. 2003-5828.

The seeding amount of the lactic acid bacteria is such that the initial number of bacteria after the seeding is 10⁵ to 10⁵ cfu/ml. When the seeding amount is less than the range, the culturing time for producing the GABA is extended. To seed more bacteria is a burden on the production of spawn.

In the step (d), the culture medium containing the mung bean extract, to which the lactic acid bacteria are seeded in the step (c) is uniformly mixed to carry out the fermentation at 20 to 35° C. When the temperature is below the range, the fermentation is not easily carried out. When the temperature is above the range, the lactic acid bacteria poor in the heat die out, so that the GABA cannot be produced.

The culturing time of the lactic acid bacteria is preferably 30 to 90 hours, and more preferably 48 to 72 hours.

A method of preparing a cosmetic composition containing a lactic acid culture of mung bean containing mung bean extract and GABA, as an effective ingredient is as follows.

The cosmetic composition containing the lactic acid culture of mung bean as an effective ingredient can be prepared according to a method comprising steps of: removing a bacterial cell and an insoluble ingredient from the lactic acid bacteria culture of mung bean containing the mung bean extract and GABA, the culture being prepared according to the method of preparing the lactic acid bacteria culture of mung bean; and mixing a sub-material with the culture from which the bacterial cell and the insoluble ingredient are removed in the preceding step.

In the step of removing the bacterial cell and insoluble ingredient from the lactic acid bacteria culture of mung bean, the bacterial cell and insoluble ingredient are removed through a treatment of activated carbon or typical filtering method (for example, filter press, membrane process and the like). Preferably, a typical filtering method is used after the treatment of activated carbon.

In particular, the bacterial cell and insoluble ingredient are subject to a decoloring process with the treatment of activated carbon. To this end, the culture is passed to a column having activated carbon packed therein or is directly mixed with the activated carbon. The invention is not particularly limited with regard to this.

In the invention, powders of activated carbon may be directly added to the culture after the culturing, so as to simplifying the processes.

In general, the activated carbon is classified into powder and particulate types depending on shapes thereof. The powder-type activated carbon is typically used with added to the liquid, for the discoloring.

The addition amount of the activated carbon is preferably 0.1 to 10 wt % of the culture, more preferably 0.5 to 5 wt %, and even more preferably 1.0 to 3.0 wt %. When the activated carbon is too much, it much costs to remove the activated carbon. When the activated carbon is too little, the discoloring is insufficient.

After the addition of the activated carbon, a heating process is immediately carried out which interrupts the fermentation of microbes and promotes the reaction of the activated carbon. The final temperature of the heating is preferably 40 to 100° C. When the final temperature is below 40° C., the microbes used for the fermentation are not extinct, so that the reaction time is prolonged. In addition, when the final temperature is above 100° C., a reverse reaction may occur.

The culture heated is left alone for 3 to 24 hours while being stirred so that the activated carbon is not settled. When it is under 3 hours, the sufficient discoloring is not achieved. When it is above 24 hours, it becomes a burden on the process. After that, the bacterial cell and insoluble ingredient are removed with the typical filtering method (for example, filter press, membrane filter, etc.).

In addition, with a method comprising a step of mixing sub-material with the lactic acid culture of mung bean having bacterial cell and insoluble ingredient removed therefrom, a cosmetic composition packed into respective formulations is prepared.

The lactic acid bacteria culture of mung bean containing the mung bean extract and GABA, which is obtained by the method of the invention, has higher effects of promoting the collagen synthesis and alleviating the skin inflammation, as compared to a prior mung bean product used for the cosmetics, and can be used for the cosmetics applied to the senescence prevention and anti-inflammation. In addition, when compared to an existing product containing mung bean extract or lactic acid bacteria culture of mung bean, it can be seen that the lactic acid bacteria culture of mung bean containing the mung bean extract and GABA, which is developed by the invention, has many advantages.

In order to examine whether the lactic acid bacteria culture of mung bean containing the mung bean extract and GABA, which is obtained using the mung bean, is actually useful for collagen secretion, the inventors conducted a Sircol Assay method. As a result, it was confirmed that when the 0.01% lactic acid bacteria culture of mung bean containing the mung bean extract and GABA was treated, the collagen secretion was promoted in a same degree as the collagen secretion induced when 10 μM genistein (Sigma, USA) was treated which was used as a positive control group. This shows that the collagen secretion was promoted by 30% or more, as compared to a non-treatment group.

In addition, in order to examine whether the lactic acid bacteria culture of mung bean containing the mung bean extract and GABA, which is obtained using the mung bean, is actually useful for the inflammation alleviation, the inventors induced inflammation in a test animal and then treated it with the culture. As a result, it was confirmed that when the 0.5% lactic acid bacteria culture of mung bean containing the mung bean extract and GABA, the inflammation was alleviated.

Accordingly, the invention provides a cosmetic composition containing the lactic acid bacteria culture of mung bean containing the mung bean extract and GABA, as an effective ingredient.

The cosmetic composition of the invention may contain other ingredients providing a synergetic effect to the main effects, in a range not deteriorating the main effects of the invention.

In addition, the cosmetic composition may take a form of solution, emulsion, viscous mixture and the like.

Although the cosmetic composition of the invention is not particularly limited with regard to its formulations, it may be formulated into skin adhesion type cosmetics, such as latex, toilet water, cream, lotion, essence, pack and gel, cosmetics having formulations such as powder, lipstick, make-up base and foundation, and washing cosmetics such as shampoo, rinse, body cleanser, cosmetic solution, cleansing foam, cleansing cream, cleansing water and soap, for example.

In the cosmetic composition of each formulation, the other ingredients may be appropriately selected and mixed by a skilled in the art, depending on formulations or use purposes of the cosmetics.

In addition, the cosmetic composition of the invention may comprise a composition selected from a group consisting of water soluble vitamin, oil soluble vitamin, high molecular peptide, high molecular polysaccharide, sphingo lipid and extract of seaweeds.

The cosmetic composition of the invention may be mixed with other ingredients which are typically mixed in the cosmetics depending on the necessities, in addition to the essential ingredients.

In addition, oil and fat ingredient, moisturizer, emollient agent, surfactant, organic and inorganic cosmetics, organic powders, ultraviolet absorbent, antiseptic, sterilizer, antioxidant, plant extract, pH conditioner, alcohol, pigment, perfume, blood stream promoter, cold sense agent, anhydrotics, purified water and the like may be added.

Further, the other ingredients may be added in a range not deteriorating the objects and effects of the invention, preferably in an amount of 0.01˜0.5 wt %, more preferably 0.01˜3 wt %.

### MODE FOR INVENTION

Hereinafter, the invention will be more specifically described with reference to embodiments and experimental examples. However, it can be understood by a skilled in the art that these embodiments and experimental examples are provided to specifically illustrate the invention, not to limit it.

### Embodiments 1 to 4

### Preparations of a Lactic Acid Bacteria Culture of Mung Bean Containing Mung Bean Extract and GABA and a Cosmetic Composition Containing the Same

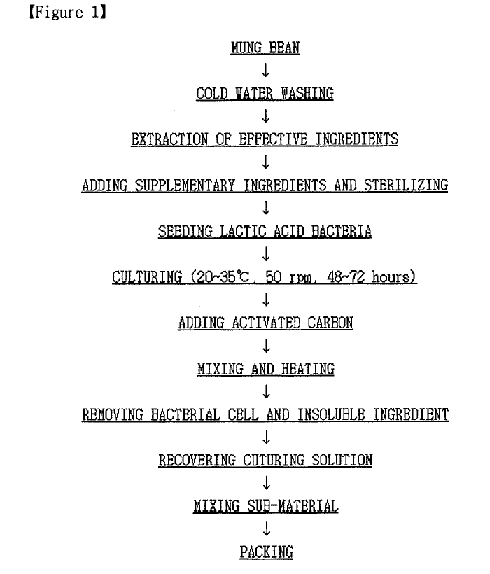

In following embodiments 1 to 4, it was prepared a lactic acid bacteria culture of mung bean containing mung bean extract and GABA and a cosmetic composition containing the same as an effective ingredient (refer to FIG. 1).

### Embodiment 1

### Preparation of Mung Bean Extract

The mung bean was washed with cold water of 30° C. or less and pulverized. Then, 100 g of mung bean was added with water, which is extraction solvent, and then extracted at 60° C. for 12 hours. After that, the supernatant solution was removed through a centrifugal separation (6000 rpm, 15 minutes), so that 800 g of mung bean extract was obtained.

### Embodiment 2

### Preparation of Lactic Acid Bacteria Culture of Mung Bean

The 500 g of mung bean extract, which was obtained in the embodiment 1, was mixed with 500 g of culture medium having ingredients and contents shown in a Table 1. The mixture was heated for sterilization at 80° C. for 30 minutes to prepare a culture medium for culturing lactic acid bacteria and including the mung bean. Then, Lactobacillus sakei B2-16 (Deposit No. KFCC-11321) (refer to a Korean Patent Application No. 2003-5828) as the lactic acid bacteria was seeded in the culture so that the initial number of bacteria was 10⁷ to 10⁸ cfu/ml. Then, the culture was cultured at 30° C. for 60 hours while being stirred at 50 rpm. As a result, a lactic acid bacteria culture of mung bean containing mung bean extract and GABA was obtained.

TABLE 1

| Ingredients | Contents (w/w) |
|---|---|
| carbon source (sucrose) | 4% |
| nitrogen source (yeast extract) | 2% |
| micro element (sodium acetate) | 0.1% |
| surfactant (polvsorbate) | 0.1% |
| MSG | 5% |
| purified water | 88.8% |

### Embodiment 3

### Treatment of Lactic Acid Bacteria Culture of Mung Bean

To the lactic acid bacteria culture of mung bean containing mung bean extract and GABA which was obtained in the embodiment 2 was added 2.5 wt % of activated carbon (Shingi Chemistry) and then heated to 70° C. The mixture was stirred and left alone for 10 hours so that the activated carbon was not settled. Then, the bacterial cell and insoluble ingredients were removed with a filter press. As a result, it was recovered the lactic acid bacteria culture of mung bean containing mung bean extract and GABA, from which the bacterial cell and insoluble ingredient were removed.

### Embodiment 4

### Preparation of a Cosmetic Composition Containing the Lactic Acid Bacteria Culture of Mung Bean

The lactic acid bacteria culture of mung bean containing mung bean extract and GABA, from which the bacterial cell and insoluble ingredient were removed, was mixed with sub-materials shown in following formulation examples depending on desired formulation types, thereby preparing cosmetic compositions.

### Experimental Example 1

### Whether GABA is Contained or not

In order to examine whether the lactic acid bacteria culture of mung bean prepared in the embodiment 2 contains the GABA, the culture as a sample was analyzed with a reversed-phase HPLC (Waters) with regard to the GABA content thereof.

The analysis conditions using the reversed-phase HPLC was established with reference to reports of Ibolya et al. First, the sample was subject to the centrifugal separation at 8000 rpm for 10 minutes and the supernatant solution was filtered with a membrane filter and then diluted in the distilled and deionized water in a proper concentration. The prepared sample was subject to the reversed-phase HPLC after derivatization of a free-column reaction using o-phthaldialdehyde (OPA). The OPA solution (pH 9.3) was prepared by mixing 5.0 μl of methanolic OPA, 20 ml of borate buffer solution (pH 9.9) and 50 μl of 2-mercaptoethanol. The methanolic OPA was prepared by dissolving 2.56 g of OPA in 50 ml of methanol and the borate buffer solution was prepared by mixing 0.2M boric acid and 0.2M sodium hydroxide in a ratio of 50:50 (v/v) and then adding 0.2M potassium chloride thereto. The OPA solution was used after 2 hours and was stable for 1 week after the preparation. The prepared OPA solution 380 μl and the sample 120 μl were mixed well and then reacted at the room temperature for 8 minutes. At this time, if it is left alone at the room temperature for a long time, peak shape or area of the reversed-phase HPLC may be different due to instability of the derivative. Accordingly, the reversed-phase HPLC was carried out without leaving it alone for 1-2 hours or more at the room temperature. Then, the derivatized sample 20 μl was introduced in the column.

As the HPLC column, a XTerra column (Waters: RP 18 5 m, 4.6 mm×150 mm) was used. As mobile phases, 0.05M sodium acetate (pH 7.2) was used as a solvent A, and a mixture (pH 7.2) in which 0.1M sodium acetate, acetonitrile (HPLC grade) and methanol (HPLC grade) were mixed in a ratio of 46:44:10 (v/v/v) was used as a solvent B. Concentration gradients of the mobile phases were as follows: starting the analysis with the solvent A being 100%, the solvent B was made to be 100% after 30 minutes, the solvent B was made to be 100% until after 40 minutes, the solvent A was made to be 100% again until after 45 minutes and the solvent A was made to be 100% until after 60 minutes. The flow rate of the mobile phase was fixed to be 1 ml/min. and the GABA was detected with a U.V. detector. Under these conditions, the holding time of GABA and glutamate were 21.01 and 9.89 minutes, respectively and a limit concentration of the detection was 0.1 mM. In addition, if the concentration of the GABA exceeds 10 mM, since it exceeds the upper limit of the detector, it was diluted to correspond to it and then measured. At this time, 99% GABA from Sigma which was commercialized for reagent was used as a standard material.

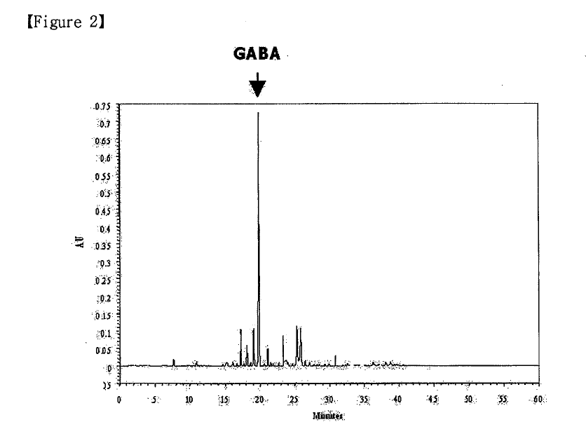

As can be seen from FIG. 2, it was confirmed that there was GABA.

### Experimental Example 2

### Collagen Synthesis Promoting Effect of Lactic Acid Bacteria Culture of Mung Bean

In order to examine the collagen synthesis promoting effect of the lactic acid bacteria culture of mung bean containing the mung bean extract and GABA, which was obtained in the embodiment 2, the Sircol Assay method was used. All collagen has a structure of [Gly-X-Y]n. According to the sircol assay method, it is used the dye reagent which is peculiarly coupled to such portion to measure the collagen in a manner of quantifying the coupled structure of collagen and dye reagent.

First, the cell lines used to measure the collagen was fibroblast NIH 3T3 cell lines (ATCC, USA) and maintained while being cultured in a DMEM culture medium (FBS 10%). In testing, in the DMEM culture medium, 4×10⁴ cells were divided in each of 24-well plates. Each of the well plates was filled with 1 ml of cell culture. At this time, before treating the first material, 500 μl of cell culture was divided therein and cultured for 12 hours, for adhesion of the cells.

In addition, in order to the culture to be used for each treatment group, as a positive control group, genistein (Sigma, USA), which was known to have an excellent collagen synthesis effect, was dissolved in DMSO. The treatment concentrations of the lactic acid bacteria culture of mung bean in the embodiment 2 and the genistein were 0.005%, 0.01% and 0.10% and 0.5 μM, 1 μM and 10 μM, respectively.

The lactic acid bacteria culture of mung bean in the embodiment 2 and the genistein were divided in the 24-well plates that were cultured for 12 hours, by 500 μl, respectively, and then cultured for 24 hours. After the culturing, 800 μl of supernatant solution was obtained and subject to the centrifugal separation at 12000 g, so that it was obtained the culture except the cells.

In order to quantify the overall water soluble collagen, the culture was centrifugally separated and then supernatant solution was obtained which was then used as a measurement sample. First, the water soluble collagen provided to obtain a quantitative standard curve was divided in standard solution with regard to concentrations thereof, and the culture to be tested was divided in 1.5 ml tubes by 500 μl, with 4 groups. The supernatant solution was mixed with Sirius red dye 1 ml which was provided from Sircol assay (Biocolor, N. Ireland) and then left alone at the room temperature for 30 minutes. In order to obtain compounds of collagen and dye, the mixture was subject to the centrifugal separation for 5 minutes as 16,000 g, washed with ethanol two times and then again dissolved in 1 ml of 5N NaOH. This was moved into 96-well plates in a proper amount and then absorbency thereof was measured at 540 nm for quantification with a visible/ultraviolet absorption measuring device.

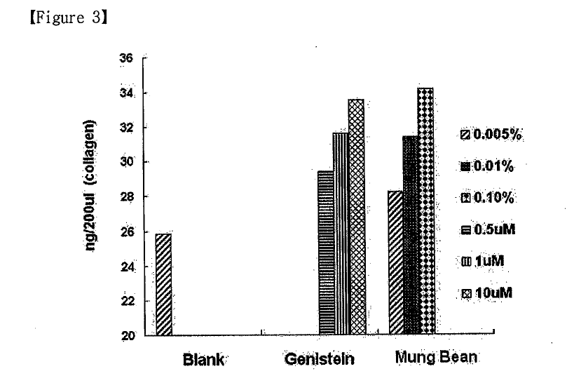

As a result, as can be seen from FIG. 3, it was confirmed that when the 0.005% lactic acid bacteria culture of mung bean (shown as mung bean in FIG. 3) was treated, the collagen secretion was further promoted, as compared to the case where the 10 μM genistein (Sigma, USA) used as the positive control group was treated.

### Experimental Example 3

### Inflammation Alleviating Effect of TPA

In order to examine the inflammation (skin stimulus) alleviating effect of the lactic acid bacteria culture of mung bean containing the mung bean extract and GABA, which was obtained in the embodiment 2, a following test was conducted according to a method used by Sheu et al. (The Journal of Investigative Dermatology, 118, pp 94-101, 2002).

First, CD-1 mouse (male & female, 6-8 weeks, Charles River, USA) were used as test objects, and as stimulus material, 10 μl of TPA (12-0-tetradecanonylphorbol-13-acetate) 0.03% (wt/vol. in acetone) solution was applied to inside and outside of both ears of the test animals. Due to the treatment material, the ear was thickened and there occurred an inflammatory reaction. After that, as a positive control group, clofibrate (1 mM) and Wy14643 (1 mM) which are agonists of PPAR α (Sheu et al., The Journal of Investigative Dermatology, 118, pp 94-101, 2002), and as a test material, the lactic acid bacteria culture of mung bean in the embodiment 2 were applied to the both surfaces of the ears in an amount of 30 ml/cm², respectively, at 45 minutes and 4 hours after the inflammation induction.

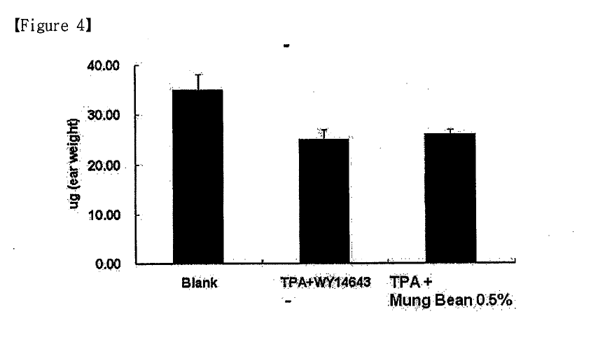

As a result, as can be seen from FIG. 4, as compared to the control group (blank) treated with TPA only, in the treatment group of TPA and WY14643 (shown as TPA+WY14643 in FIG. 4) and the treatment group of TPA and 0.5% lactic acid bacteria culture of mung bean in the embodiment 2 (shown as TPA+0.5% in FIG. 4), the swelling was decreased and the decrease effect was much higher in the latter.

In addition, in order to obtain a more detailed cytological finding, it was fixed to 4% formaldehyde and dyed with H&E. Then, the inflammation alleviation was observed with a photograph obtained by magnifying the H&E dyed tissue slide by 100 times.

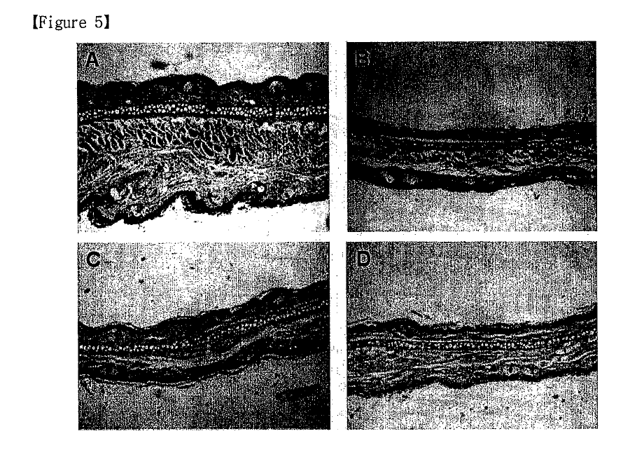

As a result, as can be seen from FIG. 5, it was confirmed that the tissue was considerably thickened in the control group (blank) treated with TPA only. However, in the treatment group D of the lactic acid bacteria culture of mung bean of the embodiment 2, the inflammation was significantly alleviated. In addition, it was confirmed that the inflammation was also alleviated in the treatment group C of Wy14643 and in the treatment group B of clofibrate, which are agonists of PPAR α.

### Experimental Example 4

### Effect on Interleukin-6 Secretion in the Skin Keratinocyte after Ultraviolet Illumination

In order to examine an effect of the lactic acid bacteria culture of mung bean containing mung bean extract and GABA, which was obtained in the embodiment 2, on secretion of interleukin-6 (IL-6) related to anti-inflammatory and hyperimmune reactions, a following test was conducted.

First, 5×10⁴ cells/well of ceratinocye (HaCaT, ATCC, USA) were seeded in the 24-well plates. On the next day, the culture medium was removed, 500 μl of PBS was added, 25 mJ/cm² UVB was illuminated and then the PBS was removed. Then, the lactic acid bacteria culture of mung bean of the embodiment 2 was included in amounts of 0.01% (pGB 0.01) and 0.1% (pGB 0.1), respectively, and GABA was included as comparative material in amounts of 0.01% (GB 0.01) and 0.1% (GB 0.1), respectively. Then, they were put into the culture medium to which WY14643 200 μM whose anti-inflammatory effect was proved was added as a positive control group and then cultured for 24 hours.

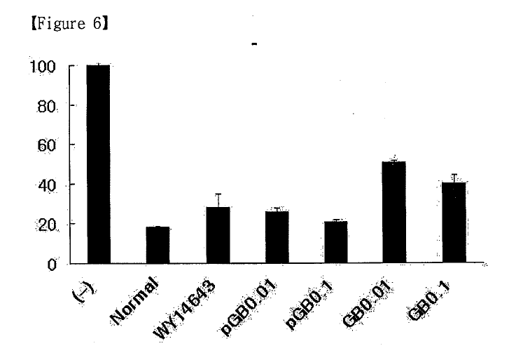

After that, in order to measure a decrease amount of IL-6, CytElisa Human IL-6 ELISA kit (USA) was used. The test was conducted as follows, according to a method provided from the maker. 100 μl of culture solution after the culturing for 24 hours was extracted and divided in 96-well plates. Then, 25 μl of anti-interleukin-6 was put in the plates which were then sealed with a plate sealer. Then, it was reacted at the room temperature for 3 hours and then washed with PBS five times. Then, goat anti-rabbit alkaline phosphatase was pun in each plate which was again sealed. Then, it was reacted at the room temperature for 45 minutes. After that, a color generating reagent was divided therein in an amount of 200 μl, respectively. Then, it was reacted at the room temperature until there occurred a color change. When there occurred the color change, a reaction terminating solution was divided in an amount of 50 μl, respectively. Then, the absorbency was measured at 490 nm to measure the content of IL-6. A result thereof is shown in FIG. 6. In FIG. 6, (−) is a group in which only vehicle was treated after the ultraviolet illumination, normal is a state before the ultraviolet illumination and the others are groups to which the materials were treated after the ultraviolet illumination, as described above.

As shown in FIG. 6, when the lactic acid bacteria culture of mung bean of the embodiment 2 was treated, it was obtained an IL-6 decreasing effect higher than WY14643 which exhibits a typical anti-inflammatory effect in proportional to the concentration. In addition, it was obtained the IL-6 decreasing effect higher than the GABA standard material. Accordingly, it was confirmed the excellent anti-inflammatory effect of the lactic acid bacteria culture of mung bean containing the mung bean extract and GABA.

In the followings, it will be described formulation examples of the composition. However, it should be noted that the examples are provided to illustrate the invention, not to limit it.

### Formulation Example 1

### Soap Preparation

lactic acid bacteria culture of mung bean of the
embodiment 3: 1.00 (wt %)

| | |
|---|---|
| oil and fat | proper amount |
| sodium hydroxide | proper amount |
| sodium chloride | proper amount |
| perfume | small amount |

Based on purified water of 100, soap was prepared according to the mixing ratio.

### Formulation Example 2

### Lotion Preparation

lactic acid bacteria culture of mung bean of the
embodiment 3: 3.00 (wt %)

| | |
|---|---|
| L-ascorbic acid-2-magnesium phosphate | 1.00 |
| water soluble collagen (1% aqueous solution) | 1.00 |
| sodium citric acid | 0.10 |
| citric acid | 0.05 |
| extract of licorice | 0.20 |
| 1,3-butyleneglycol | 3.00 |

Based on purified water of 100, lotion was prepared according to the mixing ratio.

### Formulation Example 3

### Cream Preparation

lactic acid bacteria culture of mung bean of the
embodiment 3: 1.00 (wt %)

| | |
|---|---|
| polyethyleneglycolmonostearate | 2.00 |
| self-emulsified type mono stearic acid glycerine | 5.00 |
| cetyl alcohol | 4.00 |
| squalene | 6.00 |
| trig-ethyl hexanoic acid glycerine | 6.00 |
| glycosphingolipid | 1.00 |
| 1,3-butyleneglycol | 7.00 |

Based on purified water of 100, cream was prepared according to the mixing ratio.

### Formulation Example 4

### Pack Preparation

lactic acid bacteria culture of mung bean of the
embodiment 3: 5.00 (wt %)

| | |
|---|---|
| polyvinyl alcohol | 13.00 |
| L-ascorbic acid-2-magnesium phosphate | 1.00 |
| lauroylhydroxyproline | 1.00 |
| water soluble collagen (1% aqueous solution) | 2.00 |
| 1,3-butyleneglycol | 3.00 |
| ethanol | 5.00 |

Based on purified water of 100, pack was prepared according to the mixing ratio.

### Formulation Example 5

### Toilet Water Preparation

lactic acid bacteria culture of mung bean of the
embodiment 3: 2.00 (wt %)

| | |
|---|---|
| hydroxyethylenecellulose (2% aqueous solution) | 12.00 |
| xanthan gum (2% aqueous solution) | 2.00 |
| 1,3-butyleneglycol | 6.00 |
| dark glycerine | 4.00 |
| sodium hyaluronate (1% aqueous solution) | 5.00 |

Based on purified water of 100, toilet water was prepared according to the mixing ratio.

### INDUSTRIAL APPLICABILITY

The lactic acid bacteria culture of mung bean containing the mung bean extract and GABA of the invention is able to exhibit the effect of preventing or improving the skin senescence through the collagen synthesis promotion. In addition, it can exhibit an anti-inflammatory effect and an effect of alleviating or improving the skin stimulus. Accordingly, the lactic acid bacteria culture of mung bean containing the mung bean extract and GABA can be usefully used as the cosmetic composition for promoting collagen biosynthesis, preventing or improving skin senescence, anti-inflammatory and preventing or improving skin injury, as the cosmetic composition.

1-17. (canceled)

18. A method for skin care comprising transdermally administering to a subject in need thereof a Lactobacillus sakei culture of mung bean derived from a culture medium comprising mung bean extract and monosodium glutamate (MSG),
wherein the culture medium comprises sucrose, yeast extract, sodium acetate, polysorbate, mung bean extract, MSG, and water,
wherein the amount of the MSG is 1 to 30 wt % based on a total weight of the mung bean extract,
wherein the Lactobacillus sakei culture of mung bean comprises γ-Aminobutyric acid (GABA), and
wherein the skin care comprises promoting collagen biosynthesis, preventing or treating the effects of skin senescence, anti-inflammation, or treating the effects of skin injury.

19. The method according to claim 18, wherein the Lactobacillus sakei culture of mung bean is obtained by a process comprising culturing Lactobacillus sakei in a culture medium containing the mung bean and MSG, the process comprising the steps of:
(a) preparing mung bean extract;
(b) preparing a culture medium including the mung bean extract obtained in step (a) and MSG;
(c) seeding Lactobacillus sakei in the culture medium prepared in step (b), the Lactobacillus sakei being capable of converting the MSG into GABA by glutamate dicarboxylase; and
(d) culturing the Lactobacillus sakei seeded in step (c) to prepare a lactic acid culture of mung bean containing the GABA.

20. The method according to claim 18, wherein the Lactobacillus sakei is a lactic acid bacteria expressing glutamate dicarboxylase to convert MSG into GABA.

21. The method according to claim 19, wherein the process further comprising the step of:
(e) removing a bacterial cell and an insoluble ingredient from the Lactobacillus sakei culture of mung bean containing GABA.
